(19) United States
(12) Patent Application Publication
(10) Pub. No.: US 2023/0172906 A1
(43) Pub. Date: Jun. 8, 2023

(54) METHOD TO TREAT VIRAL INFECTION INDUCED BY A CORONAVIRUS

(71) Applicants: INSERM (INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); UNIVERSITÉ PARIS CITÉ, Paris (FR)

(72) Inventors: Ralf JOCKERS, Paris (FR); Erika CECON, Paris (FR); Julie DAM, Paris (FR)

(21) Appl. No.: 17/997,978

(22) PCT Filed: May 4, 2021
(86) PCT No.: PCT/EP2021/061744

§ 371 Date: Nov. 4, 2022

(30) Foreign Application Priority Data
May 5, 2020 (EP) 20305442.4

(51) Int. Cl. A61K 31/4045; A61K 31/343; A61K 31/165; A61P 31/14; A61P 29/00

(52) U.S. Cl. CPC A61K 31/4045; A61K 31/343; A61K 31/165; A61P 31/14; A61P 29/00

(57) ABSTRACT

The present invention relates the treatment of a viral infection and particularly, the treatment of Covid-19. Melatonin (MLT) and melatoninergic drugs synchronize biological rhythms and sleep patterns, modulate the immune response, modulate the cellular oxidative status and favor cell survival in the lung under stress and inflammatory conditions. In addition to the possible mechanisms of action mentioned above, additional putative mechanism of MLT against SARS-CoV-2 comes from data of the inventors on evaluating the docking of MLT into the solved crystal structure of ACE2 with the viral protein, which revealed a possible interaction of MLT with ACE2 in close proximity to the viral interaction surface. These results suggested that the prophylactic and/or acute treatment with MLT and MLT-derived approved drugs like Ramelteon and Agomelatine, alone or associated with anti-viral or other treatments, could be of therapeutic value. Thus, the present invention relates to a melatoninergic compound for use in the treatment of a viral infection in a subject in need thereof.

### FIELD OF THE INVENTION

The present invention relates to a melatoninergic compound for use in the treatment of a viral infection in a subject in need thereof.

### BACKGROUND OF THE INVENTION

The current worldwide sanitary crises caused by the rapid spread of COVID-19 infection is an immense challenge for the health system and requires innovative solutions to be solved. At this moment there is no specific treatment to prevent or to cure COVID-19 infection, respectively. While vaccine and drug development programs are extremely long processes, repurposing of safe and already approved drugs is emerging as an additional alternative either as a prophylaxis to protect against an upcoming infection wave and to protect risk populations like nurses in contact with infected patients or to treat COVID-19 patients at the different disease states in order to increase the survival rate while decreasing the number of severe patients in need of intensive care hospitalization.

The main complications observed in COVID-19-infected patients are respiratory failure, excessive pro-inflammatory response (cytokine storm) and exhaustion and cell death of the immunological cells producing anti-viral antibodies. Evidence for viral brain invasion and substantial neurological and psychiatric morbidity in the 6 months after COVID-19 infection have also been reported. Apart from impairing the virus entry into the cells, compounds able to regulate the inflammatory response and to favor the adaptive immune response by boosting the production of anti-viral antibodies by T cells should be relevant candidates to treat COVID-19 patients. In parallel, drugs able to prevent or limit the COVID-19-associated neurological disorders will be required.

Melatonin (MLT) is a natural hormone produced during the night and is involved in the synchronization of biological rhythms and sleep. MLT acts through a variety of targets, mainly through high affinity interaction with two membrane receptors—MT1 and MT2 (Jockers et al., 2016). Additional targets comprise enzymes like calmodulin, quinone reductase 2 and mitochondrial proteins, which render MLT a highly versatile molecule displaying a wide range of cellular effects that maintain the normal physiological activities of the body (Liu et al., 2019). Melatoninergic drugs currently available on the market are Ramelteon (Rozerem®, Takeda), Agomelatonin (Valdoxan®, Servier), Tasimelteon Hetlioz® (Vanda) and Circadin® (Neurim Pharmaceuticals). They are indicated for insomnia, «jet-lag », and depression. These drugs have been proven to be extremely safe and to display few or no side-effects (de Bodinat et al., 2010, McElroy et al., 2011).

More, additional beneficial effects of MLT can be suspected, such as on viral entry into cells. SARS-CoV-2, the virus of the COVID-19 pathology, enters into human cells by interacting with the extracellular domain (ECD) of the Angiotensin-II converting enzyme 2 (ACE2) and particularly via the binding of the RBD domain of the virus to ACE2 (Wang et al., 2020). The ECD can be proteolytically cleaved off, thus decreasing viral entry. This cleavage is modulated by calmodulin (CaM) (Lambert et al., 2008). Inhibition of CaM promotes ACE2 cleavage. MLT has been reported to inhibit CaM activity (Soto-Vega et al., 2004) leading to the hypothesis that it might also promote ACE2 cleavage, and thus decrease viral entry. A recent systems-pharmacology study is based on this assumption. This study generated drug-target networks of more than 2000 FDA-approved or experimental drugs and correlated them with the COVID-19/human network of interacting proteins. However, for the moment, the effect of MLT and CaM inhibition on viral entry has not been reported in the literature and melatoninergic drugs has not been currently indicated for any viral disease and the effect of MLT or melatoninergic drugs on COVID-19 patients or related viral diseases has not been reported. However, thanks to its large spectrum of action, Melatonin could be beneficial for the prevention/treatment or co-treatment of COVID-19.

### SUMMARY OF THE INVENTION

MLT and melatoninergic drugs synchronize biological rhythms and sleep patterns (often dysregulated in intensive care patients), modulate the immune response (decrease of pro-inflammatory cytokines and inflammasome activity, increases phagocytosis by macrophages to limit bacterial infections, favors lymphocytes humoral immune response) (Kim et al., 2012, Pires-Lapa et al., 2013, Ramos et al., 2018, Zarezadeh et al., 2019), modulate the cellular oxidative status and favor cell survival in the lung under stress and inflammatory conditions (Lee et al., 2009, Shokrzadeh et al., 2015).

In addition to the possible mechanisms of action mentioned above, additional putative mechanism of MLT against SARS-CoV-2 comes from data of the inventors on evaluating the docking of MLT into the solved crystal structure of ACE2 with the viral protein, which revealed a direct interaction of MLT with ACE2 in close proximity to the viral interaction surface. This data reinforced the hypothesis that MLT might impair the virus entry in the target cell by directly interacting with ACE2. These results suggested that the prophylactic and/or acute treatment with MLT and MLT-derived approved drugs like Ramelteon and Agomelatine, alone or associated with anti-viral or other treatments, could be of therapeutic value.

Thus, the present invention relates to a melatoninergic compound for use in the treatment of a viral infection in a subject in need thereof. Particularly, the invention is defined by its claims.

### DETAILED DESCRIPTION OF THE INVENTION

A first aspect of the invention relates to a melatoninergic compound for use in the treatment of a viral infection in a subject in need thereof.

In a particular embodiment, the invention relates to a melatoninergic compound for use in the treatment of a viral infection induced by a coronavirus in a subject in need thereof.

Particularly, a melatoninergic compound could be very useful against virus cell invasion, respiratory failure, excessive pro-inflammatory response (cytokine storm), exhaustion and cell death of the immunological cells induced by the coronavirus and especially the SARS-CoV-2.

Thus, in other words, the invention also relates to a melatoninergic compound for use in the inhibition/deletion of virus cell invasion, respiratory failure, excessive pro-inflammatory response (cytokine storm), exhaustion and cell death of the immunological cells, brain invasion and neurological disorders induced by a coronavirus in a subject in need thereof.

The melatoninergic compound could be very useful to diminish viral load, in particularly in the lung and the brain.

The invention also relates to a melatoninergic compound for use in preventing the neurological effects induced by a viral infection in a subject in need thereof.

In a particular embodiment, the viral infection can be a viral lung infection, a viral brain infection, a viral heart infection or a viral skin infection. Thus, the invention also relates to a melatoninergic compound for use in the improvement of the functions of the lung, the heart, the brain or the skin after a viral infection in a subject in need thereof.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “coronavirus” has its general meaning in the art and refers to any member of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. In particular, the coronavirus particle comprises at least the four canonical structural proteins E (envelope protein), M (membrane protein), N (nucleocapsid protein), and S (spike protein). The S protein is cleaved into 3 chains: Spike protein S1, Spike protein S2 and Spike protein S2′. Production of the replicase proteins is initiated by the translation of ORF1a and ORF1ab via a −1 ribosomal frame-shifting mechanism. This mechanism produces two large viral polyproteins, pp1a and pp1ab, that are further processed by two virally encoded cysteine proteases, the papain-like protease (PLpro) and a 3C-like protease (3CLpro), which is sometimes referred to as main protease (Mpro). Coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric coV (ATCC accession #VR-1475), human coV 229E (ATCC accession #VR-740), human coV OC43 (ATCC accession #VR-920), Middle East respiratory syndrome-related coronavirus (MERS-Cov) and SARS-coronavirus (Center for Disease Control), in particular SARS-CoV-1 and SARS-CoV-2.

According to the invention, the coronavirus can be a MERS-CoV, SARS-CoV, SARS-CoV-2 or any new future family members.

As used herein, the term “melatoninergic compound” or melatoninergic drug” or “melatoninergic ligand” (in singular or plural) denotes a compound which directly modulate the melatonin system in the body or brain. Particularly, a melatoninergic compound will act through its antioxidant property or through its ability to bind a melatonin binding protein such as melatonin MT1 and MT2 receptors, CaM, ACE2, etc. (see for example Liu et al., 2019). A melatoninergic compound according to the invention can be selected in the group consisting in but not limited to melatonin (Circadin®), Ramelteon (Rozerem®), Agomelatonin (Valdoxan®), Tasimelteon (Hetlioz®) TIK-301, PD-6735, LY-156735, β-methyl-6-chloromelatonin (see for example Zlotos et al. 2014 and Jockers et al. 2016).

Tests for determining the capacity of a compound to be a melatoninergic compound are well known to the person skilled in the art, as described in Carocci et al. Clin Pharmacol. (2014) and Liu et al. Front Endocrinol (2019).

Melatoninergic compounds may be determined by any competing assay well known in the art. For example, the assay may consist in determining the ability of the compound to bind to melatonin binding proteins such as melatonin MT1 and MT2 receptors, CaM, ACE2 etc. The binding ability is reflected by the determination of the Ki using binding methods. The term “Ki”, as used herein, is intended to refer to the inhibition constant, which is calculated from the experimentally derived IC50 (half maximal inhibitory concentration) value using Graph-Pad PRISM™ according to the Cheng-Prusoff equation: Ki=IC50/1+[L]/KD (Cheng & Prusoff, 1973) where L is the concentration and KD is the dissociation constant of the labeled (radiolabeled or fluorescently labeled) melatonin. Ki values for binding biomolecules can be determined using methods well established in the art.

The functional assays may be envisaged such as evaluating the ability to directly modulate the effect of melatonin on cellular systems expressing transfected or endogenous melatonin binding proteins or different tissues in the body or brain. Examples of functional assays for melatonin binding proteins may be as follows (melatonin receptors: Legros et al. Pharmacol Res Pespect (2019); quinone reductase 2: Janda et al. Mol Pharmacol (2020); CaM: Benitez K G et al. Brain Res (1991), Pozo D et al. J Cell Biochem (1997), Leon J et al. Mol Pharmacol (2000), Benitez K G et al. Biochim Biophys Acta (1996)

In some embodiment, the melatoninergic compound is a melatonin agonist.

In some embodiment, the melatoninergic compound is a compound which bind to melatonin binding proteins such as melatonin MT1 and MT2 receptors, CaM or ACE2.

In some embodiment, the melatoninergic compound binds to ACE2.

In some embodiment, the melatoninergic compound interferes with the binding of Spike protein to ACE2.

As used herein, the term “ACE2”, also known as angiotensin-converting enzyme 2 has its general meaning in the art and refers to a membrane receptor expressed on the surface of airway epithelial cells and other cells. ACE2 serves as the entry point into cells for some coronaviruses, including HCoV-NL63, SARS-CoV, and SARS-CoV-2, because it binds with the Spike protein of coronaviruses.

As used herein, the term “spike protein” or “protein S” refers to the coronaviruses spike glycoprotein that binds its cellular receptor (i.e. ACE2), and mediates membrane fusion and virus entry.

According to the invention, the term “subject” denotes a mammal, such as a rodent, a feline, a canine, and a primate. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant. Particularly, the subject denotes an human with a viral infection, particularly induced by a coronavirus. In a particular embodiment the subject is an human with co-morbidities and in the elderly.

In some embodiments, the subject can be symptomatic or asymptomatic. As used herein, the term “asymptomatic” refers to a subject who experiences no detectable symptoms for the coronavirus infection. As used herein, the term “symptomatic” refers to a subject who experiences detectable symptoms of viral lung infection and particularly coronavirus infection. Symptoms of coronavirus infection include: fatigue, anosmia, headache, cough, fever, difficulty to breathe.

In a particular embodiment, the melatoninergic compound of the invention can be administrated orally, intra-nasally, parenterally, intraocularly, intravenously, intramuscularly, intrathecally, or subcutaneously to subject in need thereof.

In particular embodiment, the melatoninergic compound of the invention is administrated by systemic administration.

As used herein, the term “systemic administration” has its general meaning in the art and refers to a route of administration of medication into the circulatory system so that the entire body is affected.

Ideally, the melatoninergic compound is administrated to the subject in prevention, especially to subjects displaying co-morbidities or to elderly or to healthy professionals and hospital staff before the apparition of the symptoms of the viral lung infection, or as an early treatment after, for example 5, 6 or 7 days after the infection. The melatoninergic compound can also be administrated to the subject in prevention and for cure of neurological disorders at early treatment and also during a longer time period (several weeks and months) after viral infection.

In specific embodiments, it is contemplated that the compound of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa).

The invention also relates to a method for treating a viral infection in a subject in need thereof comprising administering to said subject in need thereof a therapeutically effective amount of a melatoninergic compound.

In a particular embodiment, the melatoninergic compound can be used in combination with others drugs used to treat a viral infection in a subject in need thereof.

Thus, in another embodiment, the invention relates to i) melatoninergic compound, and ii) a drug used to treat a viral infection, as a combined preparation for simultaneous, separate or sequential use in the treatment of a viral infection in a subject in need thereof.

For example, the drug used to treat a viral infection may be selected in the group consisting in bronchodilators like β2 agonists and anticholinergics, corticosteroids, beta2-adrenoceptor agonists like salbutamol, anticholinergic like ipratropium bromide or adrenergic agonists like epinephrine. Further agent may be also an antiviral compound like amantadine, rimantadine, pleconaril, azitromicine, ivementine or chloroquine.

### Therapeutic Composition

A second object of the invention relates to a therapeutic composition comprising a melatoninergic compound for use in the treatment of a viral infection in a subject in need thereof.

In a particularly embodiment, the invention relates to a therapeutic composition comprising a melatoninergic compound for use in the treatment of a viral infection induced by a coronavirus in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, intrathecal, parenteral, intraocular, intravenous, intramuscular, hippocampal stereotactic or subcutaneous administration and the like.

In particular embodiment, the pharmaceutical compositions of the invention is formulated for systemic administration.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent useful to treat a viral infection or the symptoms induced by the viral lung infection. For example, further agent may be selected in the group consisting in bronchodilators like β2 agonists and anticholinergics, corticosteroids, beta2-adrenoceptor agonists like salbutamol, anticholinergic like ipratropium bromide or adrenergic agonists like epinephrine. Further agent may be also an antiviral compound like amantadine, rimantadine, pleconaril, azitromicine, ivementine or chloroquine.

The invention will be further illustrated by the following figures and examples. However, these examples and figures should not be interpreted in any way as limiting the scope of the present invention.

### FIGURES

FIG. 1: Melatonin inhibits the binding of RBD to hACE using the TR-FRET-based assay. (A) Competition of fluorescently labeled (d2 fluorephore) RBD binding to Lumi4-Tb-SNAP-ACE2 in HEK293 cells by the indicated melatonin concentrations. Non-specific binding is defined in the presence of an excess of unlabeled RBD (1 μM). Data are expressed as mean±SEM of at 5 independent experiments, each performed in triplicate. **p<0.01, ***p<0.005 by one-way ANOVA test followed by Sidak multiple comparison post-test. (B) Determination of cell toxicity of melatonin in HEK293 cells at the indicated concentrations.

FIG. 2: In vitro effect of melatonin on SARS-CoV-2 pseudovirus entry into human HEK293 cells expressing human ACE2. Data are expressed as mean±SEM of 4 independent experiments. *p<0.05 by Student t test.

FIG. 3: Figure legend: Study of the effect of melatoninergic compounds for COVID-19 treatment in vivo in transgenic mouse model expressing human ACE2 -body weight, clinical score and survival. (A) Body weight was monitored daily (post infection) over 7 days (**p<0.01 by two-way ANOVA test followed by Dunnett post-test). (B) Body weight loss was expressed as % of mice with more than 5% body weight loss at day 7 post infection. (C) The clinical score was determined twice per day from day 4 to 7. Results are expressed as % of mice with a clinical score >9 out of 14. (D) Kaplan-Meier plot of survival curve.

FIG. 4: Viral RNA Load in lung and brain. (A). Viral RNA levels in the lung of SARS-CoV-2 infected mice. Data are expressed as mean±SEM. *p<0.05 by one-way ANOVA with two-stage linear step-up procedure of Benjamini, Krieger and Yekutieli as post-test for multiple comparisons. (B). Repartition of infected mice according to viral levels in the lungs. (C). Repartition of infected mice according to viral levels in the brain. (D). Ratio of viral load in the brain over the viral load in the lung.

FIG. 5: Cytokine levels in the lung. (A). Repartition of SARS-CoV-2 infected mice according to high level of cytokine mRNA and high viral load in the lungs 7 days after infection. Distribution values for each cytokine is cumulated in the graph. The order of cytokines in the legend reflects the order in the graph. (B-C). Level of type I interferons mRNA in the lung, IFNa (B) and IFNb (C).

### EXAMPLE

Material & Methods

Melatonin, Agomelatine and Ramelteon were purchased from Abcr company (Karlsruhe, Germany).

Docking Studies:

Docking of melatonin (PDB Chem: ML1) to the crystal structure of ACE2 in complex with the S protein of SARS-CoV-2 virus (PBD: 6M17) was performed using the AutoDock Vina open-source program (Trott & Olson, 2010) using the following center coordinates: 175.529; 114.59; 249.511; without constrains.

Cell Survival Assay

The viability of human cell lines (HEK293 and Vero cells) are assessed by Cell Titer Blue reagent (Promega, Wis., USA).

Cytokine Levels and Viral Load

RNA from frozen lung of non-infected and infected mice is extracted with TRIzol Plus RNA Purification Kit. RNA is reverse transcribed using the Maxima 1str cDNA Synth kit and qPCR is performed using the Taqman fast advance mix and Taqman cytokine primers and taqman HPRT. Viral load in the lung is measured by assessing SARS-CoV-2 ORF1 RNA. Brain RNA is extracted from ReliaPrep™ FFPE Total RNA Miniprep System from fixed brain and N1 and N2 primer/probe sets are used to measure N gene of SARS-CoV-2.

Spike S protein—hACE2 Interaction Assay

SNAP-tagged ACE2 is fluorescently labeled by incubating the cells with a SNAP suicide substrate conjugated to the long-lived fluorophore Terbium cryptate (Tb; Lumi4-Tb, 100 nM; Cisbio Bioassays, France) in Tag-lite labeling medium (1 h, on ice) (Keppler et al., 2003). After several washes, cells are collected using enzyme-free cell dissociation buffer (Sigma-Aldrich), resuspended in Tag-lite buffer and distributed into a 384-well plate. Efficient fluorescent labeling of SNAP is verified by reading fluorescence signal at 620 nm. Cells are then incubated with vehicle or melatonin for 1 h, followed by addition of RBD-d2 (receptor binding domain of the SARS-CoV-2 Spike protein labeled with the d2 fluorophore), all diluted in Tag-lite buffer. After 2 h incubation, TR-FRET signal is detected using a plate reader (Tecan F500; Tecan, Männedorf, Switzerland) with the following settings: excitation at 340 nm (Tb, energy donor), emission at 665 nm (d2, acceptor) and 620 nm (donor); delay of 150 μs; and integration time of 500 μs.

Murine Models

The inventors use the K18-hACE2 transgenic mice expressing human ACE2 in airway epithelial cells driven by a human cytokeratin 18 (K18) promoter (Jackson Laboratory, https://www.jax.org/strain/034860) (McCray et al., 2007, Yang et al., 2007, Yoshikawa et al., 2009).

Treatment Protocol

Mice are i.p. injected with melatoninergic ligands daily, one hour before lights off (in order to avoid disturbing the natural daily rhythm of MLT production), starting 2 days before virus inoculation. Mice are anesthetized with halothane and are infected via nasal inoculation of virus (10 uL each nostril, 10⁴ PFU) in Dulbecco's modified Eagle medium. Treatment is continued until the end of the experiment (7 days maximum). Mice die 7 days after inoculation at the latest. Conditions: näive (no virus), Vehicle, MLT (10 or 50 mg/kg), ramelteon (10 mg/kg), agomelatine (20 mg/kg). 6 mice/condition=24 mice in total. Plasma, lung and brain samples are taken directly after sacrifice.

Survival Rate, Clinical Score

Infected mice are examined and weighed daily. An IACUC approved clinical scoring system was utilized to monitor disease progression and establish human endpoints. Categories evaluated included body weight, posture/fur, activity/mobility, eye closure, respiratory rate are evaluated twice per day and defined as clinical score according to Moreau et al; 2020 (Am. J. Trop. Med. Hyg., 103(3), 2020, pp. 1215-1219) with a maximal score of 14. Mice died either from natural death or were scarified for ethical reasons when reaching a clinical score of 5 for 2 parameters and for 2 consecutive observation periods, or if weight loss was equal to or greater than 20%.

Cellular Entry of SARS-CoV-2 Pseudovirus

The effect of melatoninegic compounds on SARS-Cov-2 entry into host cells was investigated using a SARS-CoV-2 pseudotyped lentivirus comprising a lentivirus expressing the SARS-CoV-2 protein in the envelope and a firefly luciferase reporter gene (BPS Bioscence). Human cells (HEK293) are transfected to express ACE2, and 24 h post-transfection cells are pre-incubated with melatoninergic compounds (1 h) followed by incubation with the SARS-CoV-2 pseudovirus (1:250 dilution) for 6 h. Cells are then washed and luciferase signal is measured 48 h later by adding the firefly luciferase substrate (Promega).

Results

Docking Studies:

The inventors performed docking studies to test the hypothesis of a direct interaction between MLT and ACE2, which could impact the viral entry into cells. Results reveal a direct interaction of MLT at an ACE2 site in close proximity to the interaction surface of ACE2 and the viral spike glycosylated protein (S protein) (data not shown).

Blind docking of MLT into the two solved crystal structures of ACE2 with the receptor binding domain of the viral spike S protein (Shang et al., 2020, Yan et al., 2020) revealed a possible interaction of MLT with ACE2, in a binding pocket and an entry channel located in close proximity to the ACE2-Spike S protein interface and distinct from the central catalytic site of ACE2 (data not shown). Importantly, R393 of ACE2 is in direct contact with Y505 of the spike S protein and with MLT providing a structural basis for the potential modulatory effect of MLT on the host-virus interaction (data not shown). Agomelatine and ramelteon, two melatonin-like drugs, bind to the same binding site, including the R393 contact, with even higher predicted binding constants than MLT (data not shown). Mutation of the R393 residue significantly modified the affinity of ACE2 for the spike S protein (Procko, 2020). Interestingly, we observed that R393 and its surrounding undergo substantial conformational changes (data not shown) upon binding to the spike S protein and upon binding of the ORE-1001 inhibitor to the catalytic cleft of ACE2 ((Towler et al., 2004). Taken together, our docking results indicate the existence of a previously unknown binding cavity of ACE2 that is explored by MLT. This cavity is distinct from the large catalytic cleft and is part of the interface with the spike S protein. MLT is predicted to interact with R393 that governs the affinity of ACE2 for the spike S protein and that is sensitive to conformational changes, a property that might be relevant to modify the binding of the spike S protein upon MLT binding.

Melatonin Inhibits the Binding of RBD to hACE Using the TR-FRET-Based Assay

The inventors evaluated the effect of melatonin on the RBD/ACE2 interaction with a time-resolved Fluorescence Resonance Energy Transfer (TR-FRET) assay.

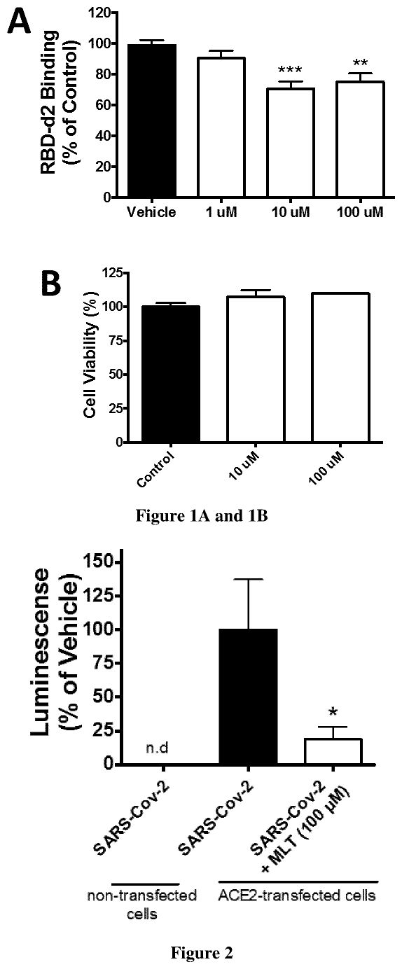

The inventors showed that melatonin at 10 and 100 μM inhibits, compared to vehicle, the binding of RBD-d2 of the SARS-CoV-2 Spike protein to the Lumi4-Tb-labeled SNAP-tagged human ACE2 (FIG. 1A) without affecting cell viability (FIG. 1B).

Study of the Effect of Melatoninergic Compounds for COVID-19 Treatment in In Vitro Models:

The inventors evaluated the effect of melatonin on the entry of SARS-CoV-2 in HEK293 cells expressing the human ACE2.

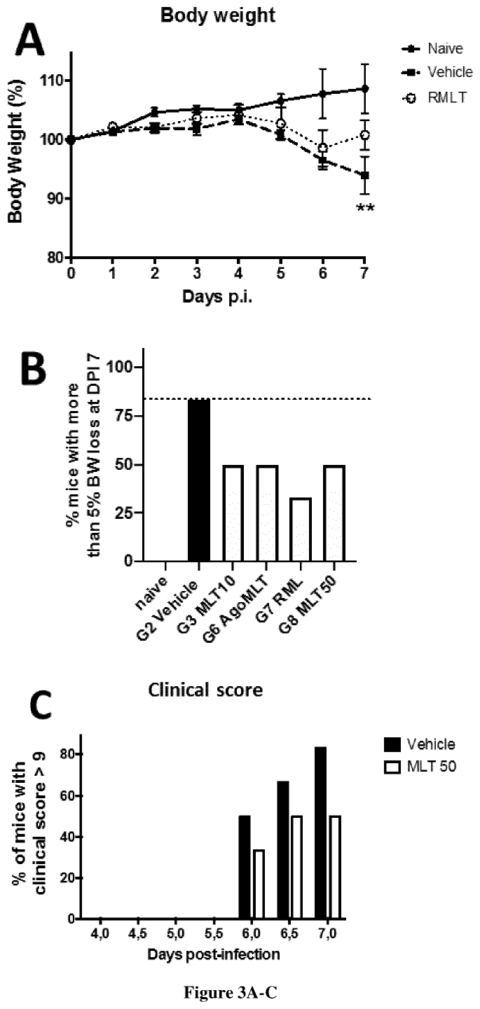

The inventors showed that only HEK293 cells expressing the human ACE2 can be infected by the SARS-CoV-2 pseudovirus. This infection could be inhibited by melatonin (FIG. 2).

Study of the Effect of Melatoninergic Compounds for COVID-19 Treatment In Vivo:

The inventors evaluated the therapeutic effect of melatoninergic ligands in the K18-hACE2 SARS-CoV-2 mouse model.

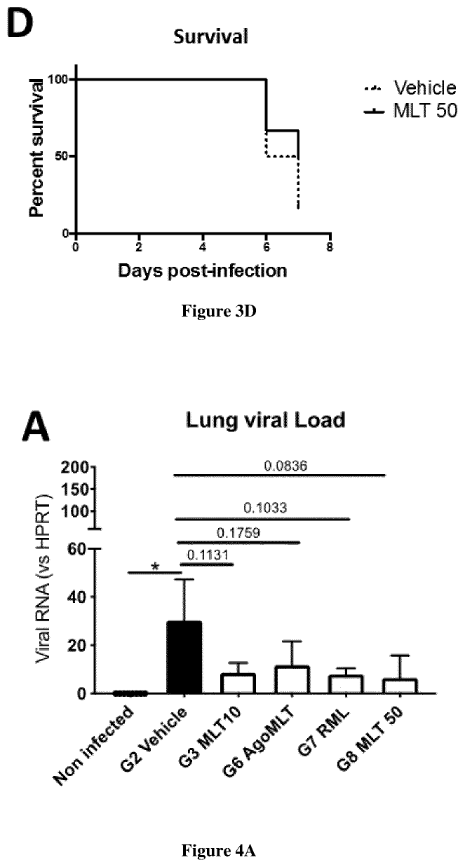

The inventors showed that melatoninergic ligands limit the body weight loss observed after SARS-CoV-2 infection (FIGS. 3A and 3B). Melatoninergic ligands improved the clinical score at the end point of the experiment (day 7 post infection) (FIG. 3C) and the cumulative score from day 4 to 7 (data not shown). Melatoninergic ligands increased the percentage of survival at the end point of the experiment (day 7 post infection) (FIG. 3D).

The inventors then evaluated the therapeutic effect of melatoninergic ligands in decreasing viral load in the lung and brain of the K18-hACE2 SARS-CoV-2 mouse model.

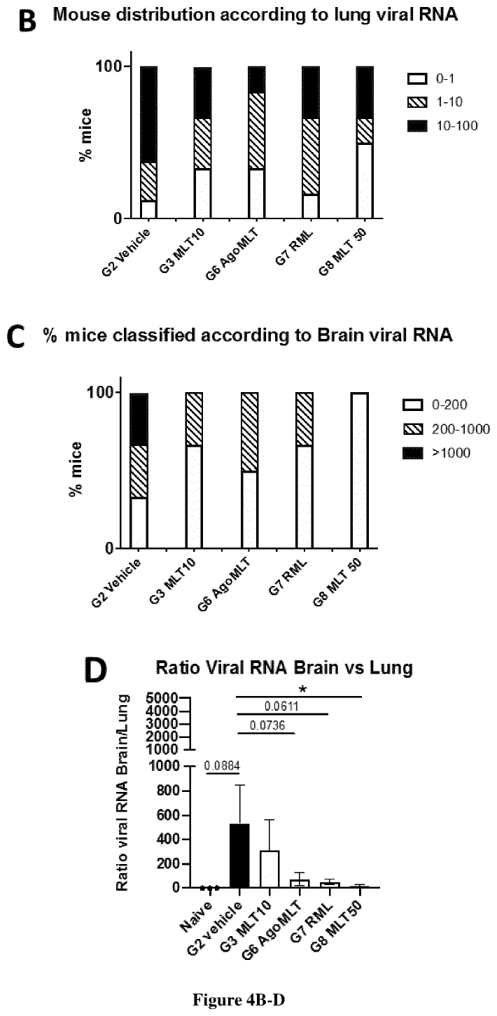

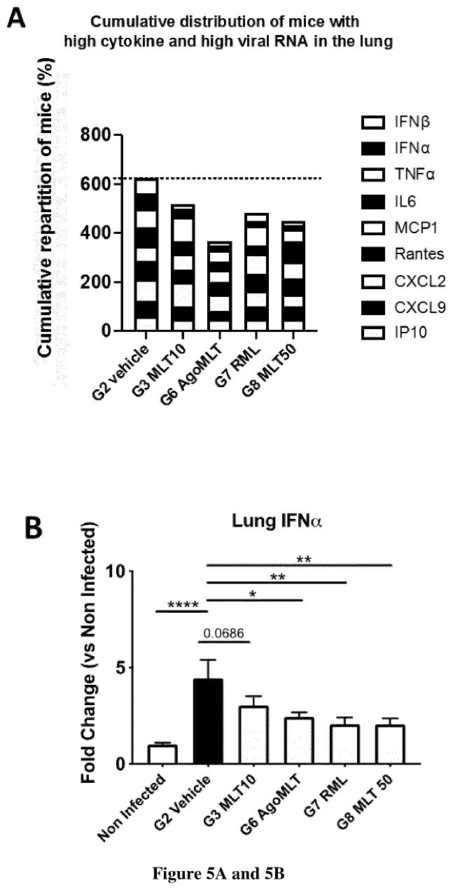

The inventors showed that melatoninergic ligands decrease the viral load in the lungs of SARS-CoV-2 infected mice after 7 days of infection (FIG. 4A). Melatoninergic ligands improved the viral load in the lungs and in the brain by limiting the number of mice with high viral load in the lungs (FIG. 4B) and high viral load in the brain (FIG. 4C). Accordingly, melatoninergic ligands decreased the ratio of brain viral RNA/lung viral RNA, suggesting protection from SARS-CoV-2 infection in these tissues.

The inventors also evaluated the therapeutic effect of melatoninergic ligands in decreasing cytokine level in the lung of the K18-hACE2 SARS-CoV-2 mouse model.

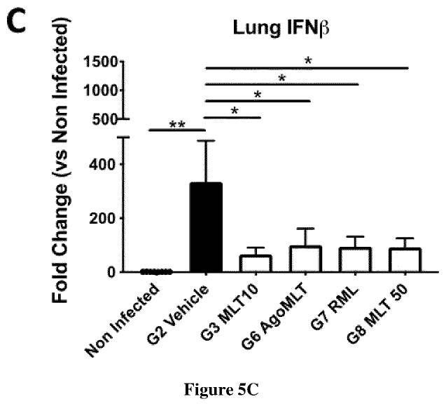

The inventors showed that melatoninergic ligands limit the overall levels of cytokines in the lungs of SARS-CoV-2 infected mice (FIG. 5A). Melatoninergic ligands decreased the detrimental high level of type I interferons (IFNa and IFNb) in the lungs, 7 days after SARS-Cov-2 infection (FIG. 5. B-C).

Thus, the inventors showed a therapeutic improvement with MLT derived drug treatment in the infected SARS-Cov-2 mouse model. They also demonstrated that melatoninergic drugs diminishes viral load particularly in the brain suggesting that melatoninergic ligands could be beneficial in preventing the long-term neurological effects of SARS-Cov-2 infection. These results provide strong evidences to support the repurposing of melatoninergic drugs to treat covid-19 patients.

### REFERENCES

Throughout this application, various references describe the state of the art to which this invention pertains. The disclosures of these references are hereby incorporated by reference into the present disclosure.

Benitez K G, Huerto D L, Anton T F. Melatonin modifies calmodulin cell levels in MDCK and N1E-115 cell lines and inhibits phosphodiesterase activity in vitro. Brain Res. (1991) 557:289-92.

Benitez K G, Rios A, Martinez A, Anton T F. In vitro inhibition of Ca2+/calmodulin-dependent kinase II activity by melatonin. Biochim Biophys Acta. (1996) 1290:191-6.

Carocci et al. Melatonergic drugs in development. Clin Pharmacol. 2014; 6: 127-137.

Cheng Y, Prusoff W H. Relationship between the inhibition constant (K1) and the concentration of inhibitor which causes 50 percent inhibition (I50) of an enzymatic reaction. Biochem Pharmacol. 1973. 22: 3099-108

de Bodinat C, Guardiola-Lemaitre B, Mocaer E, Renard P, Munoz C, Millan M J (2010) Agomelatine, the first melatonergic antidepressant: discovery, characterization and development. Nat Rev Drug Discov 9: 628-642

Janda E, Nepveu F, Calamini B, Ferry G, Boutin J A. Molecular Pharmacology of NRH:Quinone Oxidoreductase 2: A Detoxifying Enzyme Acting as an Undercover Toxifying Enzyme. Mol Pharmacol. 2020 November; 98(5):620-633.

Jockers R, Delagrange P, Dubocovich M L, Markus R P, Renault N, Tosini G, Cecon E, Zlotos D P (2016) Update on Melatonin Receptors. IUPHAR Review. Br J Pharmacol 173: 2702-2725

Kim J Y, Lee Y D, Kim B J, Kim S P, Kim D H, Jo K J, Lee S K, Lee K H, Baik H W (2012) Melatonin improves inflammatory cytokine profiles in lung inflammation associated with sleep deprivation. Mol Med Rep 5: 1281-4

Lambert D W, Clarke N E, Hooper N M, Turner A J (2008) Calmodulin interacts with angiotensin-converting enzyme-2 (ACE2) and inhibits shedding of its ectodomain. FEB S Lett 582: 385-90

Lee Y D, Kim J Y, Lee K H, Kwak Y J, Lee S K, Kim O S, Song D Y, Lee J H, Baik T K, Kim B J, Kim J Y, Baik H W (2009) Melatonin attenuates lipopolysaccharide-induced acute lung inflammation in sleep-deprived mice. J Pineal Res 46: 53-7

Legros C, et al. Characterization of the various functional pathways elicited by synthetic agonists or antagonists at the melatonin MT(1) and MT(2) receptors. Pharmacol Res Perspect. 2019 Dec. 29; 8(1):e00539. doi: 10.1002/prp2.539.

Leon J, Macias M, Escames G, Camacho E, Khaldy H, Martin M, et al. Structure-related inhibition of calmodulin-dependent neuronal nitric-oxide synthase activity by melatonin and synthetic kynurenines. Mol Pharmacol. (2000) 58:967-75. doi: 10.1124/mol.58.5.967

Liu L, Labani N, Cecon E, Jockers R (2019) Melatonin Target Proteins: Too Many or Not Enough? Front Endocrinol (Lausanne) 10: 791

McCray P B, Jr., Pewe L, Wohlford-Lenane C, Hickey M, Manzel L, Shi L, Netland J, Jia H P, Halabi C, Sigmund C D, Meyerholz D K, Kirby P, Look D C, Perlman S (2007) Lethal infection of K18-hACE2 mice infected with severe acute respiratory syndrome coronavirus. J Virol 81: 813-21

McElroy S L, Winstanley E L, Martens B, Patel N C, Mori N, Moeller D, McCoy J, Keck P E, Jr. (2011) A randomized, placebo-controlled study of adjunctive ramelteon in ambulatory bipolar I disorder with manic symptoms and sleep disturbance. Int Clin Psychopharmacol 26: 48-53

Pires-Lapa M A, Tamura E K, Salustiano E M, Markus R P (2013) Melatonin synthesis in human colostrum mononuclear cells enhances dectin-1-mediated phagocytosis by mononuclear cells. J Pineal Res 55: 240-6

Pozo D, Reiter R J, Calvo J R, Guerrero J M. Inhibition of cerebellar nitric oxide synthase and cyclic GMP production by melatonin via complex formation with calmodulin. J Cell Biochem. (1997) 65:430-42. doi: 10.1002/(SICI)1097-4644(19970601)65:3<430::AID-JCB12>3.0.CO; 2-J

Procko E. The sequence of human ACE2 is suboptimal for binding the S spike protein of SARS coronavirus 2. bioRxiv. 2020

Ramos A, Miguez M P, Morgado S, Sanchez-Correa B, Gordillo J J, Casado J G, Tarazona R, Regodon S (2018) Melatonin enhances responsiveness to Dichelobacter nodosus vaccine in sheep and increases peripheral blood CD4 T lymphocytes and IgG-expressing B lymphocytes. Vet Immunol Immunopathol 206: 1-8

Shang J. et al. Structural basis of receptor recognition by SARS-CoV-2. Nature. 581, pages 221-224(2020

Shokrzadeh M, Chabra A, Naghshvar F, Ahmadi A, Jafarinejhad M, Hasani-Nourian Y (2015) Protective Effects of Melatonin against Cyclophosphamide-induced Oxidative Lung Toxicity in Mice. Drug Res (Stuttg) 65: 281-6

Soto-Vega E, Meza I, Ramirez-Rodriguez G, Benitez-King G (2004) Melatonin stimulates calmodulin phosphorylation by protein kinase C. J Pineal Res 37: 98-106

Towler P. et al. ACE2 X-ray structures reveal a large hinge-bending motion important for inhibitor binding and catalysis. J Biol Chem. 2004 Apr. 23; 279(17):17996-8007.

Trott O, Olson A J. J Comput Chem. 2010 Jan. 30; 31(2):455-61. doi: 10.1002/jcc.21334. PMID: 19499576

Wang Q, Zhang Y, Wu L, Niu S, Song C, Zhang Z, Lu G, Qiao C, Hu Y, Yuen K Y, Wang Q, Zhou H, Yan J, Qi J (2020) Structural and Functional Basis of SARS-CoV-2 Entry by Using Human ACE2. Cell

Yan R. et al. Structural basis for the recognition of SARS-CoV-2 by full-length human ACE2. Science (2020):Vol. 367, Issue 6485, pp. 1444-1448

Yang X H, Deng W, Tong Z, Liu Y X, Zhang L F, Zhu H, Gao H, Huang L, Liu Y L, Ma C M, Xu Y F, Ding M X, Deng H K, Qin C (2007) Mice transgenic for human angiotensin-converting enzyme 2 provide a model for SARS coronavirus infection. Comp Med 57: 450-9

Yoshikawa N, Yoshikawa T, Hill T, Huang C, Watts D M, Makino S, Milligan G, Chan T, Peters C J, Tseng C T (2009) Differential virological and immunological outcome of severe acute respiratory syndrome coronavirus infection in susceptible and resistant transgenic mice expressing human angiotensin-converting enzyme 2. J Virol 83: 5451-65

Zarezadeh M, Khorshidi M, Emami M, Janmohammadi P, Kord-Varkaneh H, Mousavi S M, Mohammed S H, Saedisomeolia A, Alizadeh S (2019) Melatonin supplementation and pro-inflammatory mediators: a systematic review and meta-analysis of clinical trials. Eur J Nutr

Zlotos D P, Jockers R, Cecon E, Rivara S, Witt-Enderby P A (2014) MT1 and MT2 Melatonin Receptors: Ligands, Models, Oligomers, and Therapeutic Potential. J Med Chem 57: 3161-3185

1. A method of treating a viral infection in a subject in need thereof, comprising
administering to the subject a therapeutically effective amount of a melatoninergic compound.

2. The method according to claim 1 wherein the viral infection is a viral lung infection, a viral brain infection, a viral heart infection or a viral skin infection.

3. The method according to claim 1 wherein the viral infection is induced by a coronavirus.

4. The method according to claim 3 wherein the coronavirus is MERS-CoV, SARS-CoV or the SARS-CoV-2.

5. The method according to claim 4 wherein the coronavirus is SARS-CoV-2.

6. The method according to claims 1 wherein the melatoninergic compound is melatonin, Ramelteon, Agomelatine, Tasimelteon, TIK-301, PD-6735, LY-156735 or the β-methyl-6-chloromelatonin.

7. (canceled)

8. The method of claim 1, wherein the method comprises simultaneous, separate or sequential administration of the melatoninergic compound and a drug used to treat a viral infection.

9. (canceled)

10. A method of inhibiting one or more of virus cell invasion, respiratory failure, excessive pro-inflammatory response, exhaustion and cell death of immunological cells, brain invasion and neurological disorders induced by a coronavirus in a subject in need thereof, comprising
administering to the subject a therapeutically effective amount of a melatoninergic compound.
